(19) United States
(12) Patent Application Publication
(10) Pub. No.: US 2023/0210459 A1
(43) Pub. Date: Jul. 6, 2023

(54) Wearable Biosignal Measurement Element and Biosignal Measurement Device

(71) Applicant: Nippon Telegraph and Telephone Corporation, Tokyo (JP)

(72) Inventors: Yoshiyuki Doi, Tokyo (JP); Toshishige Shimamura, Tokyo (JP); Yuzo Ishii, Tokyo (JP); Satoshi Tsunashima, Tokyo (JP)

(21) Appl. No.: 18/000,787

(22) PCT Filed: Jun. 29, 2020
(86) PCT No.: PCT/JP2020/025493

§ 371 Date: Dec. 5, 2022

(51) Int. Cl. A61B 5/00; A61B 5/25

(52) U.S. Cl. CPC A61B 5/6804; A61B 5/25; A61B 2562/0209

(57) ABSTRACT

A wearable biological signal measuring element is attached to a garment and detachably connected to a controller, the wearable biological signal measuring element including: an electrode in contact with skin; an electric line connected to the electrode; an electric connector connected to the electric line, penetrating the garment, and detachably connected to the controller outside the garment; and a water supply mechanism including a water guiding mechanism and a first water tank connected to an opening of the water guiding mechanism inside the garment and including a through hole in a contact surface with the electrode.

### CROSS-REFERENCE TO RELATED APPLICATIONS

This application is a national phase entry of PCT Application No. PCT/JP2020/025493, filed on Jun. 29, 2020, which application is hereby incorporated herein by reference.

### TECHNICAL FIELD

The present invention relates to a wearable biological signal measuring element and a biological signal measuring device capable of making measurement with high accuracy for a long period of time.

### BACKGROUND

In recent years, for an electrode in a wearable device that measures a biological signal such as a heart rate signal or an electrocardiographic signal, a conductive polymer or metal having particularly excellent conductivity and hydrophilicity is used as a material having good compatibility with a living body.

This wearable device is used by being embedded in a garment, but when contact resistance (impedance) increases due to drying of an electrode portion in contact with skin, a waveform deteriorates in a biological signal to be measured. The change in the resistance value causes waveform distortion and noise increase in a desired signal, and causes signal disappearance (lead-off state). Therefore, in order to maintain the contact resistance, it is necessary to maintain an appropriate wet environment of the electrode.

A basic configuration and operation of a biological signal measuring device 90 disclosed in Non Patent Literature 1 will be described with reference to FIGS. 12 and 13.

FIG. 12 is a front view of a garment 1 to which the biological signal measuring device 90 is attached. Broadly, the biological signal measuring device 90 includes the garment 1, electrode portions 91, electric lines 92, and a controller 94. The plurality of electrode portions 91 is provided on an inner surface (inside) of the garment 1, which is a surface in contact with skin 2. Each of the electric lines 92 is electrically connected to a corresponding one of the electrode portions 91, and is connected to the controller 94.

FIG. 13 is a front view of the biological signal measuring device 90 as viewed from the inside of the garment 1. Furthermore, FIG. 14 is a cross-sectional view taken along line XIV-XIV′ illustrated in FIG. 13. The controller 94 is a detachable electric component, is installed outside the garment 1, and is electrically connected via electric connectors 93. Each of the electric connectors 93 is installed so as to penetrate the garment, and is connected to a corresponding one of the electric lines 92 inside the garment 1. The electrode portions 91 come into contact with the skin 2 at the time of measuring a biological signal.

Here, for the garment 1, a material such as polyester or cotton, which is used for general underwear such as a T-shirt, is used. For the electrode portions 91 and the electric lines 92, a material containing a conductive polymer or metal is used.

For each of the electric connectors 93, a component is used that serves both as a mechanism for electrically connecting to the controller 94 and a mechanism for attaching and detaching the controller 94, and for example, a snap button is used. In addition, although not illustrated in detail, the controller 94 includes a detector that detects an electric signal from a living body, an amplifier that amplifies the signal, a memory that stores the signal data, a wireless component that transmits the signal to an external device, and the like.

An electric signal (for example, an electrocardiographic waveform) detected by the electrode portions 91 is transmitted to the controller 94 via the electric lines 92 and the electric connectors 93. Although not illustrated here, the controller 94 accumulates the signal data in the memory, converts the signal data into a wireless signal, and then transmits the wireless signal to an external computer or terminal.

Next, a configuration of an electrode portion disclosed in Patent Literature 1 will be described with reference to FIG. 15. FIG. 15 is a cross-sectional view of one of the electrode portions 91 illustrated in FIG. 13 taken along line XIV-XIV′. The electrode portion 91 includes, inside the garment 1, an electrode 911 that comes into contact with the skin 2 and detects a biological signal, a base material 915 including a through hole 913, a cover 916 attached to the base material 915, and a humidity adjustment material 914 installed inside the base material 915 and the cover 916. In addition, one of the electric lines 92 is connected to the electrode 911 and transmits the electric signal to the controller 94.

A basic operation in FIG. 15 is similar to that in FIGS. 13 and 14, and the electric signal detected by the electrode portion 91 is transmitted to the controller 94 via one of the electric lines 92 and one of the electric connectors 93. The configuration illustrated in FIG. 15 has a humidity adjustment function by the humidity adjustment material 914 in order for the electrode 911 to maintain an appropriate humidity environment.

The humidity adjustment material 914 is a material that can be impregnated with a moisturizing agent such as water or glycerol, and can also absorb sweat generated on the skin.

### CITATION LIST

### Patent Literature

Patent Literature 1: Japanese Patent No. 5706575

### Non Patent Literature

Non Patent Literature 1: Tsukada, Yayoi Tetsuou et al. “Validation of wearable textile electrodes for ECG monitoring.” Heart and vessels vol. 34, 7 (2019): 1203-1211. doi: 10.1007/s00380-019-01347-8

### SUMMARY

### Technical Problem

However, in the biological signal measuring device disclosed in Non Patent Literature 1, since the electrode portions include no humidity adjustment mechanism, a problem frequently occurs that a waveform deteriorates in a biological signal to be measured when the contact resistance increases due to drying of the electrode portions in contact with the skin.

In addition, there is a problem that, in daily biological activities, waveform distortion, noise increase, and a lead-off state occur in a desired signal without being noticed when a change in the contact resistance value is measured. Here, the lead-off state refers to a state in which the waveform cannot be acquired (measured).

Furthermore, since when a user notices the drying and lead-off state and supplies water to the electrodes, an appropriate supply amount of water is unknown, there is also a problem that excessively wetting the electrodes causes physical and mental stress to a living body, and for example, a cold feeling is caused by excessive wetting of the electrodes at the time of wearing the garment, or discomfort is caused by stuffiness at the time of activity.

In addition, although the biological signal measuring device disclosed in Patent Literature 1 includes the humidity adjustment mechanism, there is a case where the humidity adjustment material itself cannot maintain sufficient humidity due to, for example, an overload biological activity or an activity in a high-temperature dry atmosphere. Therefore, there is a problem that the humidity adjustment function of the electrodes deteriorates or disappears because water cannot be supplied from the outside.

Furthermore, since the humidity adjustment material is washed together with the garment and the adjustment performance deteriorates as washing is repeated, there is a problem that the performance life of the biological signal measuring device is short.

### Solution to Problem

In order to solve the problems as described above, a wearable biological signal measuring element according to embodiments of the present invention is a wearable biological signal measuring element that is attached to a garment and detachably connected to a controller, the wearable biological signal measuring element including: an electrode that comes into contact with skin; an electric line that is connected to the electrode; an electric connector that is connected to the electric line, penetrates the garment, and is detachably connected to the controller outside the garment; and a water supply mechanism including a water guiding mechanism including a water guiding portion that includes one opening outside the garment and another opening outside the garment and is supplied with water from the one opening, the another opening being substantially in close contact with the electrode.

In addition, a wearable biological signal measuring element according to embodiments of the present invention is a wearable biological signal measuring element that is attached to a garment and detachably connected to a controller, the wearable biological signal measuring element including: an electrode that comes into contact with skin; an electric line that is connected to the electrode; an electric connector that is connected to the electric line, penetrates the garment, and is detachably connected to the controller outside the garment; and a water supply mechanism including a water guiding mechanism and a first water tank that is connected to an opening of the water guiding mechanism inside the garment and includes a through hole in a contact surface with the electrode.

### Advantageous Effects of Embodiments of Invention

According to embodiments of the present invention, it is possible to provide a wearable biological signal measuring element and a biological signal measuring device capable of suppressing drying of an electrode portion and making measurement with high accuracy for a long period of time.

### BRIEF DESCRIPTION OF THE DRAWINGS

FIG. 1 is a front view of a biological signal measuring device according to a first embodiment of the present invention.

FIG. 2 is a cross-sectional view of an electrode portion in the biological signal measuring device according to the first embodiment of the present invention.

FIG. 3 is a functional block diagram of the biological signal measuring device according to the first embodiment of the present invention.

FIG. 4 is a flowchart illustrating a procedure for retaining a wet environment of an electrode in the biological signal measuring device according to the first embodiment of the present invention.

FIG. 5 is a cross-sectional view of an electrode portion in a biological signal measuring device according to a modification of the first embodiment of the present invention.

FIG. 6 is a front view of a biological signal measuring device according to a second embodiment of the present invention.

FIG. 7 is a cross-sectional view of an electrode portion in the biological signal measuring device according to the second embodiment of the present invention.

FIG. 8 is a front view of a biological signal measuring device according to a third embodiment of the present invention.

FIG. 9 is a cross-sectional view of a controller in the biological signal measuring device according to the third embodiment of the present invention.

FIG. 10 is a functional block diagram of the biological signal measuring device according to the third embodiment of the present invention.

FIG. 11 is a flowchart illustrating a procedure for retaining a wet environment of an electrode in the biological signal measuring device according to the third embodiment of the present invention.

FIG. 12 is a diagram illustrating attachment of a conventional biological signal measuring device to a garment.

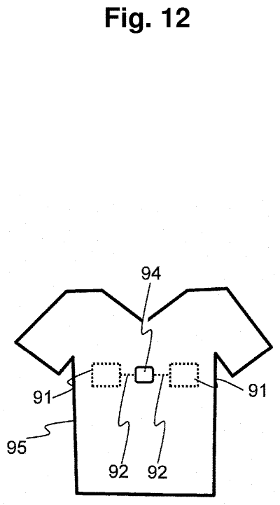

FIG. 13 is a front view of the conventional biological signal measuring device.

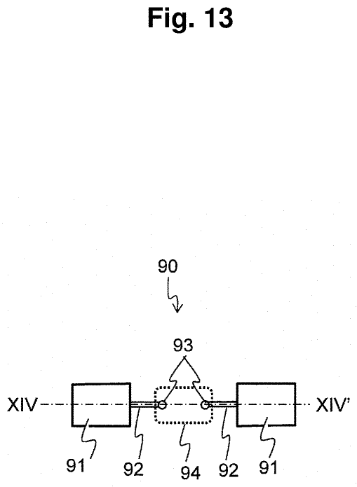

FIG. 14 is a cross-sectional view of the conventional biological signal measuring device.

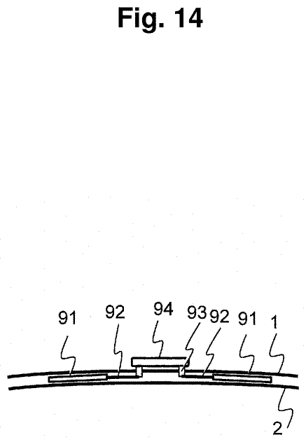

FIG. 15 is a cross-sectional view of an electrode portion in the conventional biological signal measuring device.

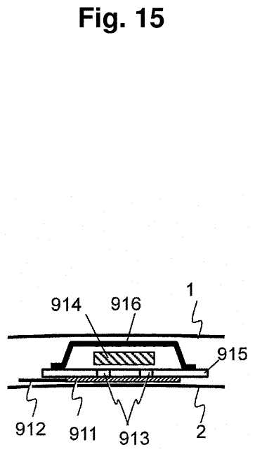

### DETAILED DESCRIPTION OF ILLUSTRATIVE EMBODIMENTS

### First Embodiment

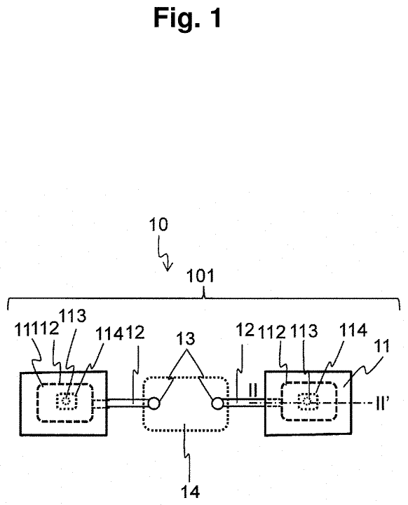

A first embodiment of the present invention will be described with reference to FIGS. 1 to 5.

<Configuration of Biological Signal Measuring Device>

FIG. 1 is a front view of a biological signal measuring device 10 according to the first embodiment as viewed from the inside of a garment 1. The biological signal measuring device 10 includes a wearable biological signal measuring element 101 and a controller 14.

The wearable biological signal measuring element 101 is attached to the garment 1, and includes electrode portions 11, electric lines 12, and electric connectors 13 inside the garment 1.

Each of the electric lines 12 is electrically connected to a corresponding one of the electrode portions 11, and is connected to the controller 14 via a corresponding one of the electric connectors 13. The electrode portions 11 are brought into contact with skin 2 at the time of measuring a biological signal.

Here, the electric connectors 13 are installed so as to penetrate the garment 1, and are attached to the detachable controller 14 (dotted line in the drawing) outside the garment 1.

With this configuration, an electric signal (for example, an electrocardiographic waveform) detected by the electrode portions 11 is transmitted to the controller 14 via the electric lines 12 and the electric connectors 13.

The controller 14 accumulates the signal data in a memory, converts the signal data into a wireless signal, and then transmits the wireless signal to an external computer or terminal.

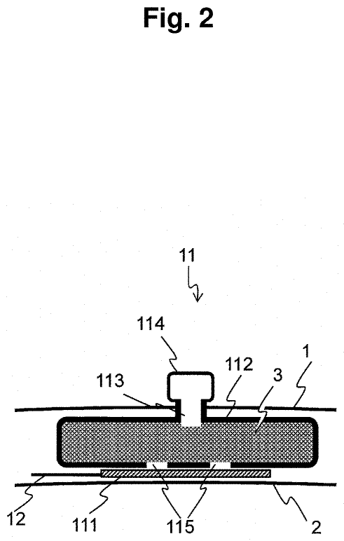

FIG. 2 is a cross-sectional view of one of the electrode portions 11 in FIG. 1 taken along line II-II′, and illustrates a configuration of the electrode portion 11. The electrode portion 11 includes, inside the garment 1, an electrode 11 that comes into contact with the skin 2 and detects a biological signal, and a water tank 112 (first water tank) that stores water.

The electrode 11 is connected to one of the electric lines 12. Here, the size of the electrode is 4 cm×3 cm, the thickness is about 5 mm, and the capacity of the water tank is about 5 ml (milliliter).

Here, the water tank 112 includes a through hole 115 on a surface in contact with the electrode 111.

A water guiding portion 113 serving as a water guiding mechanism penetrates the garment 1, and an opening of the water guiding portion 113 inside the garment 1 is connected to the water tank 112. A cap 114 is installed in an opening of the water guiding portion 113 outside the garment 1.

Water (or an aqueous solution having a moisturizing effect) 3 is supplied to the water tank 112 from the opening of the water guiding portion 113 outside the garment 1. For the water tank 112, an elastically stretchable material such as an elastomer or a rubber material is used.

As described above, the wearable biological signal measuring element 101 includes the water guiding mechanism (the water guiding portion 113) and the water tank 112 (first water tank), which serve as a water supply mechanism.

With this configuration, the water 3 supplied to the water tank 112 is supplied to the electrode 11 via the through hole 115. Furthermore, the water tank 112 stretches according to a pressing force from the outside, and a constant amount of water is supplied to the electrode 111 via the through hole 115.

In addition, for the garment 1, a material such as polyester or cotton, which is used for general underwear such as a T-shirt, is used as in the conventional apparatus.

For the electrode portions 11 and the electric lines 12, a conductive fibrous material is used as a material. As the conductive fibrous material, for example, a fiber material containing a conductive polymer or a metal can be used. For example, a material obtained by forming a film of a metal on fibers by plating, vapor deposition, or the like, or a material obtained by interweaving a metal thread into fibers can be used. As the metal, silver (Ag), aluminum (Al), or the like can be used.

For each of the electric connectors 13, a component is used that serves both as a mechanism for electrically connecting to the controller 14 and a mechanism for attaching and detaching the controller 14, and for example, a snap button is used.

Furthermore, the controller 14 includes a detector that detects an electric signal from a living body, an amplifier that amplifies the signal, a memory that accumulates the signal data, a wireless component that transmits the signal to an external device, and the like (not illustrated).

<Operation of Biological Signal Measuring Device>

An operation of the biological signal measuring device 10 according to the present embodiment will be described.

First, an electric signal detected by the electrode portions 11 is transmitted to the controller 14 via the electric lines 12 and the electric connectors 13.

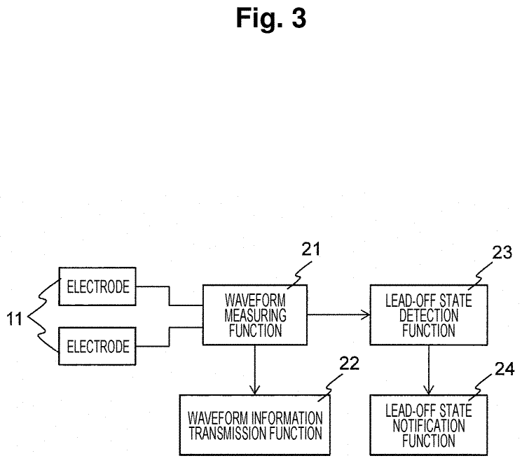

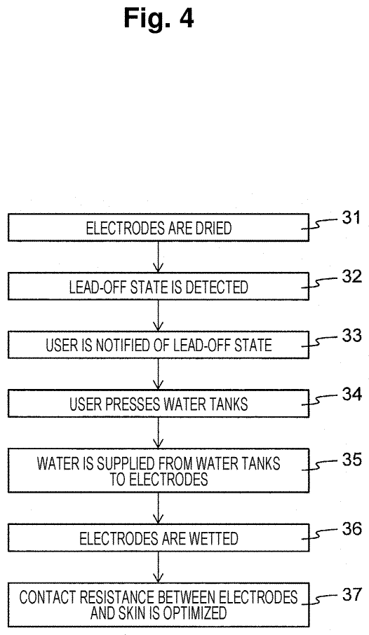

Next, a procedure for maintaining a wet environment of the electrodes 111 will be described with reference to FIGS. 3 and 4.

FIG. 3 illustrates a functional block diagram of the biological signal measuring device 10. FIG. 4 is a flowchart of the procedure for maintaining the wet environment of the electrodes 111.

A biological signal measured by a waveform measuring function 21 is transmitted from the controller 14 to an external terminal by a waveform information transmission function 22.

Meanwhile, the biological signal measured by the waveform measuring function 21 is monitored by a lead-off state detection function 23 provided in the controller 14 or the external terminal, and when the electrodes are dried, a lead-off state is detected (steps 31 and 32).

Next, a lead-off state notification function 24 notifies a device user (user) of the lead-off state by means of an alarm or the like (step 33).

Next, when the user who has received the notification pressurizes the water tanks 112 from the outside of the garment 1, the water 3 is supplied from the water tanks 112 to the electrodes 111 (steps 34 and 35).

Finally, the contact resistance between the electrodes 111 and the skin 2 is optimized by the electrodes 111 being wetted (steps 36 and 37). The above operation is performed, for example, about once a day.

Note that the notification method is provided, for example, from the external terminal (for example, a smartphone) that receives the wireless signal from the controller 14 by an alarm sound and vibration (vibration) of the terminal. Alternatively, the notification method may be provided from the controller 14 by an alarm sound and vibration (vibration) of the terminal.

According to the biological signal measuring device 10 and the wearable biological signal measuring element 101 according to the present embodiment, the electrodes 111 can be provided with an appropriate wet environment, and thus it is possible to prevent deterioration and loss of signal data.

In addition, since the lead-off state can be easily noticed in daily biological activities, it is possible to reduce waveform distortion, noise increase, and a lead-off period.

Furthermore, when the user notices the drying and lead-off state and supplies the water 3 to the electrodes 111, a supply amount of the water is clear according to the pressing force to the water tanks 112. Therefore, it is possible to appropriately wet the electrodes 111, and it is possible to reduce the occurrence of physical and mental stress to the living body, such as a cold feeling caused by excessive wetting of the electrodes 111 at the time of wearing the garment and discomfort caused by stuffiness at the time of activity.

In addition, since the humidity adjustment material disclosed in Patent Literature 1 is not required, the adjustment performance is less likely to deteriorate even in a case where the biological signal measuring device is repeatedly washed together with the garment, and the performance life of the biological signal measuring device can be extended.

Meanwhile, as in the conventional apparatus, it is also possible to enclose a humidity adjustment material in the water tanks. Here, a difference from the conventional apparatus is that the present embodiment can adjust wetting by appropriately introducing water from the outside, and thus the adjustment ability required for the humidity adjustment material in the present embodiment may be lower than the adjustment ability required for the humidity adjustment material used in the conventional apparatus. This point leads to widening of material selectability, and it is also possible to apply a humidity adjustment material having a longer life or a humidity adjustment material having excellent economic efficiency.

In addition, using an elastically stretchable material such as an elastomer or a rubber material for the water tanks 112 makes it possible to change the diameter of each of the through holes 115 when the water tanks 112 are pressurized from the outside. For example, normally, the diameter of each of the through holes 115 on the electrode side is as small as 0.1 mm, so that water is prevented from being excessively supplied to the electrodes 111.

Meanwhile, when the water tanks 112 are pressurized from the outside, the water tanks 112 stretch, and the diameter of each of the through holes 115 is increased to about 0.5 mm. In this manner, the diameter of each of the through holes 115 can be increased as necessary to change the amount of the water 3 supplied to the electrodes 111.

Note that the detected signal illustrated in the functional block diagram and the flowchart is not limited to a lead-off signal. For example, a configuration may also be possible in which a threshold value is provided for the signal intensity and a notification is issued when the signal intensity falls below the threshold value. In this case, it is possible to hold the wet environment more precisely than the detection and notification using the lead-off signal.

In the present embodiment, an example has been described in which water is supplied from the water tanks to the electrodes via the through holes, but the present invention is not limited thereto, and water can also be supplied to the electrodes via check valves disposed in the parts of the through holes. In this case, the check valves are closed normally, and are opened by pressure when the water tanks are pressurized, so that water is supplied from the water tanks to the electrodes.

<Modification of First Embodiment>

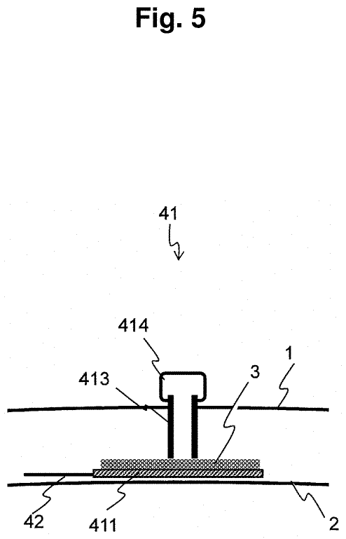

Next, a biological signal measuring device 40 according to a modification of the first embodiment will be described with reference to FIG. 5. The biological signal measuring device 40 has substantially the same configuration as the biological signal measuring device according to the first embodiment, but is different in that an electrode portion 41 of a wearable biological signal measuring element 401 includes no water tank.

FIG. 5 is a cross-sectional view of the electrode portion 41 in the wearable biological signal measuring element 401. The electrode portion 41 includes, inside the garment 1, an electrode 411 that comes into contact with the skin 2 and detects a biological signal, and a water guiding portion 413.

The electrode 411 is connected to an electric line 42.

The water guiding portion 413 penetrates the garment 1, and a cap 414 is installed in an opening of the water guiding portion 413 outside the garment 1. An opening of the water guiding portion 413 inside the garment 1 is substantially in close contact with the electrode 411.

Here, the substantially close contact means that the opening of the water guiding portion 413 is not completely in close contact with the electrode 411 but in contact to such an extent that there is a slight gap of about several mm or 1 mm or less between the opening of the water guiding portion 413 and the electrode 411, the water 3 seeps through the gap, and the electrode 411 can be supplied with the water 3.

Here, the opening of the water guiding portion 413 inside the garment 1 may be formed in a shape having an expandable diameter so that the water 3 seeping from the gap may easily spread on the surface of the electrode 411.

As described above, the wearable biological signal measuring element 401 includes the water guiding portion 413 as a water supply mechanism.

With this configuration, the water 3 injected from the opening of the water guiding portion 413 outside the garment 1 is supplied to the electrode 411.

Since the biological signal measuring device 40 includes no water tank, water cannot be retained for a long time as compared with the biological signal measuring device 10 according to the first embodiment, but the biological signal measuring device 40 can operate well if water is supplied to the biological signal measuring device 40 at an appropriate time interval and supplied to the electrode.

As described above, according to the biological signal measuring device 40 and the wearable biological signal measuring element 401 according to the present modification, water can be easily supplied to the electrode to maintain the wet state as compared with the conventional configuration without the water guiding portion.

Furthermore, in the present embodiment and modification, a small-diameter tube having an outer shape of about 0.3 mm is used as the water guiding mechanism (water guiding portion), but the present invention is not limited thereto, and for example, fibers such as hollow polyester or microchannels made of glass or metal used for heat pipes can also be applied.

### Second Embodiment

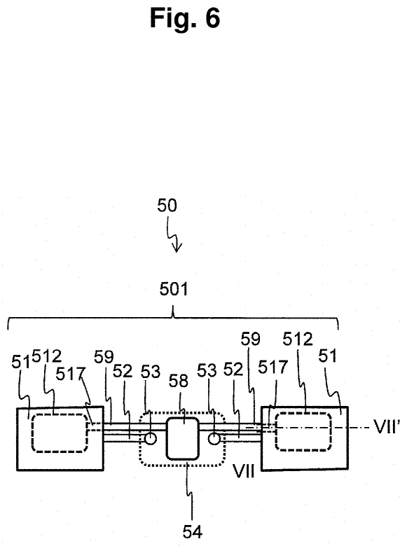

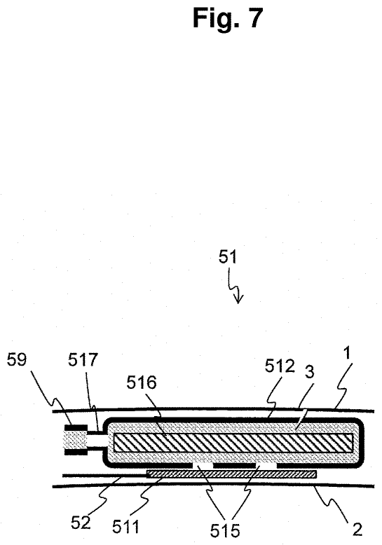

Next, a biological signal measuring device according to a second embodiment of the present invention will be described with reference to FIGS. 6 and 7.

<Configuration of Biological Signal Measuring Device>

FIG. 6 is a front view of a biological signal measuring device 50 according to the second embodiment as viewed from the inside of a garment 1. The biological signal measuring device 50 includes a wearable biological signal measuring element 501 and a controller 54.

The wearable biological signal measuring element 501 is attached to the garment 1, and includes electrode portions 51, electric lines 52, and electric connectors 53 inside the garment 1.

Each of the electric lines 52 is electrically connected to a corresponding one of the electrode portions 51, and is connected to the controller 54 via a corresponding one of the electric connectors 53. The electrode portions 51 are brought into contact with skin 2 at the time of measuring a biological signal.

Here, the electric connectors 53 are installed so as to penetrate the garment 1, and are attached to the detachable controller 54 (dotted line in the drawing) outside the garment 1.

With this configuration, an electric signal (for example, an electrocardiographic waveform) detected by the electrode portions 51 is transmitted to the controller 54 via the electric lines 52 and the electric connectors 53.

The controller 54 accumulates the signal data in a memory, converts the signal data into a wireless signal, and then transmits the wireless signal to an external computer or terminal.

Furthermore, the biological signal measuring device 50 includes a water tank 58 at a location other than the electrode portions 51. In the case of the present embodiment, the water tank (second water tank, hereinafter referred to as “control-side water tank”) 58 is installed at a position facing the controller 54 inside the garment 1, but the present invention is not limited thereto. The installation location may be a location between the position facing the controller 54 inside the garment 1 and one of the electrode portions, or may be outside the garment 1.

Furthermore, a water guiding mechanism (water passages 59) connected to the electrode portions 51 is connected to the control-side water tank 58.

In addition, the water guiding mechanism (a water guiding portion 513, which is not illustrated) is connected to the control-side water tank 58 through the garment 1, and water 3 is supplied from an opening of the water guiding mechanism (the water guiding portion 513) outside the garment 1.

In addition, the opening of the water guiding mechanism (the water guiding portion 513) outside the garment 1 is provided with a cap 514 (not illustrated).

FIG. 7 is a cross-sectional view of one of the electrode portions 51 in FIG. 6 taken along line VII-VII′, and illustrates a configuration of the electrode portion 51. The electrode portion 51 includes, inside the garment 1, an electrode 511 that comes into contact with the skin 2 and detects a biological signal, and a water tank (first water tank, hereinafter referred to as “electrode-side water tank”) 512 that stores the water 3.

The electrode 511 is connected to one of the electric lines 52. Here, the size of the electrode is 4 cm×3 cm, the thickness is about 5 mm, and the capacity of the water tank is about 5 ml (milliliter).

Here, the electrode-side water tank 512 includes a through hole 515 on a surface in contact with the electrode 511.

Furthermore, in the water guiding mechanism (a water guiding portion 517 and one of the water passages 59), an opening of the water guiding portion 517 inside the garment 1 is connected to the electrode-side water tank 512. The control-side water tank 58 is connected to an opening of the water passage 59 outside the garment 1, which is included in the water guiding mechanism (the water guiding portion 517 and the water passage 59).

For the electrode-side water tank 512 and the control-side water tank 58, an elastically stretchable material such as an elastomer or a rubber material is used.

As described above, the wearable biological signal measuring element 501 has a configuration in which the control-side water tank 58 (second water tank) is provided as a water supply mechanism in a middle portion of the water guiding mechanism, and the electrode-side water tank 512 (first water tank) is connected to the opening of the water guiding mechanism inside the garment.

With this configuration, the water 3 supplied to the control-side water tank 58 (second water tank) is supplied to the electrode-side water tank 512 (first water tank) via the water guiding mechanism, and is supplied to the electrode 511 via the through hole 515.

Furthermore, a pressing force (water pressure) to the control-side water tank 58 (second water tank) from the outside is transmitted to the electrode-side water tank 512 (first water tank), and a constant amount of the water 3 is supplied to the electrode 511 via the through hole 515 according to the water pressure.

In the present embodiment, a humidity adjustment material 516 is enclosed in the electrode-side water tank 512. The humidity adjustment material 516 is a material that can be impregnated with a moisturizing agent such as water or glycerol, and can also absorb sweat generated on the skin.

In addition, for the garment 1, a material such as polyester or cotton, which is used for general underwear such as a T-shirt, is used as in the conventional apparatus.

For the electrode portions 51 and the electric lines 52, a conductive fibrous material is used as a material. As the conductive fibrous material, for example, a fiber material containing a conductive polymer or a metal can be used. For example, a material obtained by forming a film of a metal on fibers by plating, vapor deposition, or the like, or a material obtained by interweaving a metal thread into fibers can be used. As the metal, silver (Ag), aluminum (Al), or the like can be used.

For each electric connector, a component is used that serves both as a mechanism for electrically connecting to the controller and a mechanism for attaching and detaching the controller, and for example, a snap button is used.

Furthermore, the controller includes a detector that detects an electric signal from a living body, an amplifier that amplifies the signal, a memory that accumulates the signal data, a wireless component that transmits the signal to an external device, and the like.

<Operation of Biological Signal Measuring Device>

The operation of the present device is substantially the same as that of the first embodiment, but the procedure is different in that a user who has received notification pressurizes the control-side water tank 58 instead of the electrode-side water tanks 512 when the water tank is pressurized from the outside of the garment.

As a result, the water 3 is supplied from the control-side water tank 58 (second water tank) to all the electrodes 511 via the electrode-side water tanks 512 (first water tanks) with one pressurizing operation, and the electrodes 511 are wetted, whereby the contact resistance between the electrodes 511 and the skin 2 is optimized. The above operation is performed, for example, about once a day.

According to the biological signal measuring device 50 according to the present embodiment, it is possible to provide an appropriate wet environment for the electrodes, to prevent deterioration and loss of signal data, and to recognize a lead-off state in daily biological activities, which makes it possible to reduce waveform distortion and a lead-off period.

In addition, clarifying the amount of water supplied according to the pressing force secures an appropriate wet environment to reduce the occurrence of physical and mental stress.

Furthermore, in the present embodiment, providing the control-side water tank makes it possible to reduce the operation of the user. That is, one pressurizing operation makes it possible to supply water to all the electrodes, and one introduction of water into the control-side water tank makes it possible to supply water to all the electrode-side water tanks.

Note that there are various choices of the magnitude relationship of the water tanks. For example, when the capacity of the control-side water tank is increased and the capacity of each electrode-side water tank is decreased, the weight of each electrode-side water tank can be reduced. In addition, when the capacity of the control-side water tank is decreased and the capacity of each electrode-side water tank is increased, the weight on the control side can be reduced.

Furthermore, in the present embodiment, the humidity adjustment material is installed in each electrode-side water tank, but the present invention is not limited thereto. That is, the humidity adjustment material is only required to be installed according to a required humidity adjustment performance. For example, the humidity adjustment material may be installed inside the control-side water tank. Alternatively, the humidity adjustment material does not have to be installed in any of the tanks.

In the present embodiment, since water can be appropriately introduced from the outside to adjust wetting, the adjustment ability required for the humidity adjustment material in the present embodiment may be lower than the adjustment ability required for the humidity adjustment material used in the conventional apparatus. This point leads to widening of material selectability, and it is also possible to apply a humidity adjustment material having a longer life or a humidity adjustment material having excellent economic efficiency.

Furthermore, in the present embodiment, a small-diameter tube having an outer shape of about 0.3 mm is used as the water guiding mechanism (the water guiding portion and the water passages), but the present invention is not limited thereto, and for example, fibers such as hollow polyester or microchannels made of glass or metal used for heat pipes can also be applied.

In addition, in the present embodiment, a configuration example has been described in which the electrode-side water tanks 512 (first water tanks) are provided together with the control-side water tank 58 (second water tank), but the present invention is not limited thereto. A configuration may be adopted in which the control-side water tank 58 (second water tank) is provided and the electrode-side water tanks 512 (first water tanks) are not provided. In this case, as in the modification of the first embodiment, the opening of the water guiding mechanism inside the garment is substantially in close contact with the electrode.

### Third Embodiment

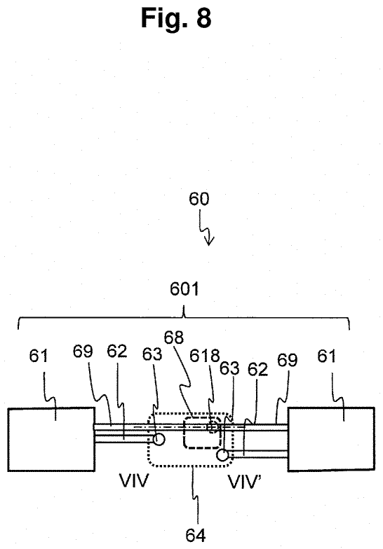

Next, a biological signal measuring device according to a third embodiment of the present invention will be described with reference to FIGS. 8 to 11.

The biological signal measuring device according to the present embodiment has substantially the same configuration as that of the second embodiment, and is different in that a control-side water tank (second water tank) is provided inside a controller. Furthermore, the present embodiment is different in that an electromagnetic valve is provided between the control-side water tank (second water tank) and electrode portions, and supply of water to the electrodes is adjusted by the electromagnetic valves. Details will be described below.

<Configuration of Biological Signal Measuring Device>

FIG. 8 is a front view of a biological signal measuring device 60 according to the third embodiment as viewed from the inside of a garment 1. The biological signal measuring device 60 includes a wearable biological signal measuring element 601 and a controller 64.

The wearable biological signal measuring element 601 is attached to the garment 1, and includes electrode portions 61, electric lines 62, and electric connectors 63 inside the garment 1.

Each of the electric lines 62 is electrically connected to a corresponding one of the electrode portions 61, and is connected to the controller 64 via a corresponding one of the electric connectors 63. The electrode portions 61 are brought into contact with skin 2 at the time of measuring a biological signal.

Here, the electric connectors 63 are installed so as to penetrate the garment 1, and are attached to the detachable controller 64 (dotted line in the drawing) outside the garment 1.

With this configuration, an electric signal (for example, an electrocardiographic waveform) detected by the electrode portions 61 is transmitted to the controller 64 via the electric lines 62 and the electric connectors 63.

The controller 64 accumulates the signal data in a memory, converts the signal data into a wireless signal, and then transmits the wireless signal to an external computer or terminal.

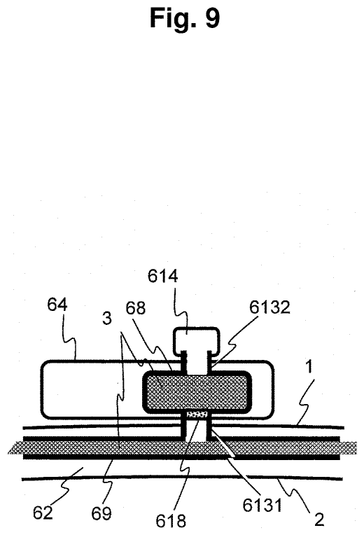

FIG. 9 is a cross-sectional view of the controller 64 in FIG. 8 taken along line VII-VII′, and illustrates a configuration of the controller 64.

The biological signal measuring device 60 includes a water tank (second water tank, hereinafter referred to as “control-side water tank”) 68 inside the controller 64.

A water guiding mechanism (a water guiding portion 6132) is connected to the control-side water tank 68 from the outside of the controller 64, and water 3 is supplied from an opening of the water guiding mechanism (the water guiding portion 6132) outside the garment 1.

In addition, the opening of the water guiding mechanism (the water guiding portion 6132) outside the garment 1 is provided with a cap 614.

Furthermore, a water guiding mechanism that supplies the water 3 to electrodes (not illustrated) of the electrode portions 61 is connected to the control-side water tank 68. The water guiding mechanism includes a water guiding portion 6131, a water passage 69, and a water guiding portion 617 (not illustrated).

One opening of the water guiding portion 6132 is connected to the control-side water tank 68 inside the controller 64, and the other opening of the water guiding portion 6132 is connected to the water passage 69 inside the garment 1.

As in the element according to the second embodiment, the water passage 69 is connected to electrode-side water tanks (first water tanks, which are not illustrated) each including a through hole in a contact surface with the electrode in a corresponding one of the electrode portions 61 via the water guiding portion (not illustrated).

Therefore, the water 3 supplied to the control-side water tank (second water tank) 68 is supplied to the electrode-side water tanks (first water tanks) through the water guiding mechanism (the water passage and the water guiding portions), and is supplied to the electrodes through the through holes of the electrode-side water tanks.

Furthermore, in the controller 64, an electromagnetic valve 618 is provided at a connection part between the control-side water tank 68 and the water guiding mechanism (the water guiding portion 6131).

The water (or an aqueous solution having a moisturizing effect) 3 is supplied from the opening of the water guiding mechanism (the water guiding portion 6132) outside the controller 64 to the control-side water tank 68. For the control-side water tank 68 and the electrode-side water tanks, an elastically stretchable material such as an elastomer or a rubber material is used.

In the biological signal measuring device according to the present embodiment, an example has been described in which the electromagnetic valve 618 is disposed at the connection part between the control-side water tank (second water tank) 68 and the water guiding mechanism (the water guiding portion 6131) inside the controller 64, but the present invention is not limited thereto. The electromagnetic valve is only required to be disposed in the water guiding mechanism between the control-side water tank (second water tank) 68 and the electrodes of the electrode portions 61. However, in consideration of simplification of electric wiring to the electromagnetic valve, the electromagnetic valve is desirably disposed near the controller.

Furthermore, in the biological signal measuring device according to the present embodiment, an example has been described in which the control-side water tank (second water tank) 68 is disposed inside the controller 64, but the present invention is not limited thereto. Even when the control-side water tank (second water tank) 68 is disposed outside the controller 64, the supply of water can be adjusted by the electromagnetic valve.

As described above, the wearable biological signal measuring element 6oi has a configuration in which the control-side water tank (second water tank) 68 is provided as a water supply mechanism in a middle portion of the water guiding mechanism, and the electrode-side water tanks (first water tanks) are connected to openings of the water guiding mechanism inside the garment.

With this configuration, the water 3 supplied to the control-side water tank (second water tank) 68 is supplied to the electrode-side water tanks (first water tanks) via opening and closing of the electromagnetic valve 618 and the water guiding mechanism, and is supplied to the electrodes of the electrode portions 61 via the through holes.

Here, the supply of water to the electrodes of the electrode portions 61 can be adjusted by the opening and closing drive of the electromagnetic valve 618.

<Operation of Biological Signal Measuring Device>

The operation of the biological signal measuring device 60 is substantially the same as that of the first and second embodiments, but is different in that the electromagnetic valve is used to adjust the supply of water to the electrodes.

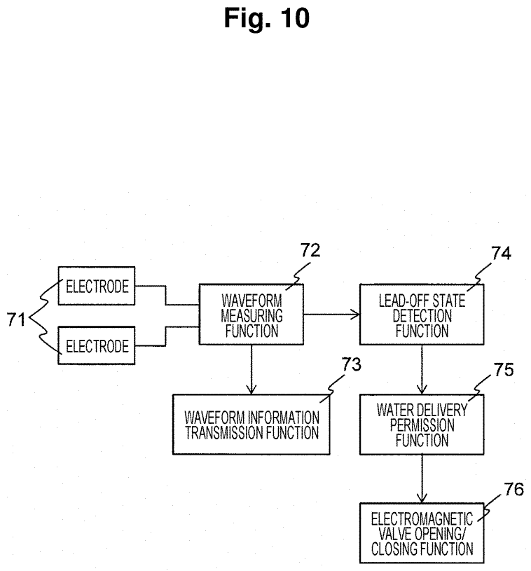

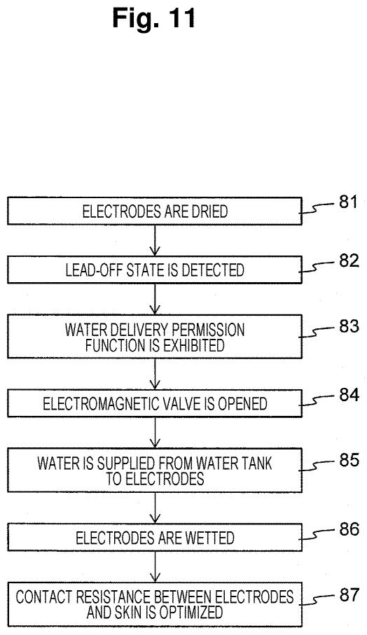

FIG. 10 illustrates a functional block diagram of the biological signal measuring device 60. FIG. 11 is a flowchart of a procedure for maintaining a wet environment of the electrodes.

A biological signal measured by a waveform measuring function 72 is transmitted from the controller 64 to an external terminal by a waveform information transmission function 73.

Meanwhile, the biological signal measured by the waveform measuring function 72 is monitored by a lead-off state detection function 74 provided in the controller 64 or the external terminal, and when the electrodes of the electrode portions 61 are dried, a lead-off state is detected (steps 81 and 82).

Next, an electromagnetic valve opening/closing function 76 is driven via a water delivery permission function 75 (steps 83 and 84).

Next, the electromagnetic valve 618 is opened by the electromagnetic valve opening/closing function 76, and the water 3 is supplied from the control-side water tank (second water tank) 68 to the electrode-side water tanks (first water tanks) via the water guiding mechanism, and is supplied to the electrodes of the electrode portions 61 via the through holes (step 85).

Finally, the contact resistance between the electrodes and the skin is optimized by the electrodes being wetted (steps 86 and 87). The above operation is performed, for example, about once a day.

According to the biological signal measuring device 60 according to the present embodiment, it is possible to provide an appropriate wet environment for the electrodes, to prevent deterioration and loss of signal data, and to recognize a lead-off state in daily biological activities, which makes it possible to reduce waveform distortion and a lead-off period.

Furthermore, clarifying the amount of water supplied according to the opening and closing operation of the electromagnetic valve secures an appropriate wet environment to reduce the occurrence of physical and mental stress.

In addition, in the present embodiment, providing the control-side water tank and the electromagnetic valve inside the controller makes it possible to supply water to all the electrodes by an electric signal without requiring a user's operation, and one introduction of water into the control-side water tank makes it possible to supply water to all the electrode-side water tanks.

Furthermore, in the present embodiment, there are various choices of the magnitude relationship of the water tanks. For example, when the capacity of the control-side water tank is increased and the capacity of each electrode-side water tank is decreased, the weight of each electrode-side water tank can be reduced. Conversely, when the capacity of the control-side water tank is decreased and the capacity of each electrode-side water tank is increased, the weight on the control side can be reduced.

Furthermore, a humidity adjustment material is only required to be installed according to a required humidity adjustment performance. For example, the humidity adjustment material may be installed inside the control-side water tank. Alternatively, the humidity adjustment material does not have to be installed in any of the tanks.

In the present embodiment, since water can be appropriately introduced from the outside to adjust wetting, the adjustment ability required for the humidity adjustment material in the present embodiment may be lower than the adjustment ability required for the humidity adjustment material used in the conventional apparatus. This point leads to widening of material selectability, and it is also possible to apply a humidity adjustment material having a longer life or a humidity adjustment material having excellent economic efficiency.

Furthermore, since the controller is not washed, the humidity adjustment material can be prevented from deteriorating by washing. This point leads to widening of material selectability, and it is also possible to apply a humidity adjustment material having a longer life or a humidity adjustment material having excellent economic efficiency.

Furthermore, in the present embodiment, a small-diameter tube having an outer shape of about 0.3 mm is used as the water guiding mechanism (the water guiding portion and the water passages), but the present invention is not limited thereto, and for example, fibers such as hollow polyester or microchannels made of glass or metal used for heat pipes can also be applied.

In addition, in the present embodiment, a configuration example has been described in which the electrode-side water tanks (first water tanks) are provided together with the control-side water tank (second water tank) 68, but the present invention is not limited thereto. A configuration may be adopted in which the control-side water tank (second water tank) 68 is provided and the electrode-side water tanks (first water tanks) are not provided. In this case, as in the modification of the first embodiment, the openings of the water guiding mechanism inside the electrode portions are substantially in close contact with the electrodes.

In each of the above embodiments of the present invention, an example has been described in which the biological signal measuring device includes the two electrode portions, but the present invention is not limited thereto, and the biological signal measuring device may include a plurality of electrode portions. Furthermore, the biological signal measuring device can operate even in the case of including one electrode portion.

In each of the above embodiments of the present invention, an example has been described in which the controller in the biological signal measuring device detects a lead-off state from a measured waveform and notifies an external terminal (for example, a smartphone) of the lead-off state, but the present invention is not limited thereto. The controller in the biological signal measuring device may transmit the measured waveform to a smartphone, a server, or the like, and the smartphone, the server, or the like may detect a lead-off state and make a notification of the lead-off state. The notification result may be output to the server or the like as an alarm sound or vibration, or may be transmitted to the biological signal measuring device (controller) and output.

In each of the above embodiments of the present invention, examples of the structure, dimension, material, and the like of each component have been described in the configuration, operation procedure, and the like of the biological signal measuring device, but the present invention is not limited thereto. Any device may be used as long as the device exerts a function and an effect of the biological signal measuring device.

### INDUSTRIAL APPLICABILITY

Embodiments of the present invention can be applied to a wearable device that measures a biological signal such as a heart rate signal or an electrocardiographic signal.

### REFERENCE SIGNS LIST

10 Biological signal measuring device
101 Wearable biological signal measuring element
11 Electrode portion
12 Electric line
13 Electric connector
14 Controller
111 Electrode
112 First water tank
113 Water guiding mechanism (water guiding portion)
114 Cap
115 Through hole.

1-8. (canceled)

9. A wearable biological signal measuring element that is attached to a garment and detachably connected to a controller, the wearable biological signal measuring element comprising:
an electrode configured to come into contact with skin;
an electric line that is connected to the electrode;
an electric connector that is connected to the electric line, penetrates the garment, and is detachably connected to the controller outside the garment; and
a water supply mechanism including a water guiding mechanism including a water guiding portion, the water guiding porting including a first opening outside the garment and a second opening outside the garment, wherein the water guiding portion is configured to be supplied with water from the first opening, and wherein the second opening is substantially in close contact with the electrode.

10. The wearable biological signal measuring element according to claim 9, further comprising a water tank in a middle portion of the water guiding mechanism.

11. The wearable biological signal measuring element according to claim 10, wherein the water guiding mechanism includes an electromagnetic valve between the water tank and the electrode.

12. The wearable biological signal measuring element according to claim 11, wherein a lead-off state is configured to be detected when the electrode is dried, and the electromagnetic valve is configured to be driven to supply water.

13. The wearable biological signal measuring element according to claim 9, wherein a lead-off state is configured to be detected when the electrode is dried.

14. The wearable biological signal measuring element according to claim 9, wherein the electrode is made of a conductive fibrous material.

15. A wearable biological signal measuring element that is attached to a garment and detachably connected to a controller, the wearable biological signal measuring element comprising:
an electrode configured to come into contact with skin;
an electric line that is connected to the electrode;
an electric connector that is connected to the electric line, penetrates the garment, and is detachably connected to the controller outside the garment; and
a water supply mechanism including a water guiding mechanism including a first water tank connected to an opening of the water guiding mechanism inside the garment, the first water tank including a through hole in a contact surface with the electrode.

16. The wearable biological signal measuring element according to claim 15, further comprising a second water tank in a middle portion of the water guiding mechanism.

17. The wearable biological signal measuring element according to claim 16, wherein the water guiding mechanism includes an electromagnetic valve between the second water tank and the electrode.

18. The wearable biological signal measuring element according to claim 17, wherein a lead-off state is configured to be detected when the electrode is dried, and wherein the electromagnetic valve is configured to be driven to supply water.

19. The wearable biological signal measuring element according to claim 15, wherein a lead-off state is configured to be detected when the electrode is dried.

20. The wearable biological signal measuring element according to claim 15, wherein the electrode is made of a conductive fibrous material.

21. A biological signal measuring device comprising:
a controller; and
a wearable biological signal measuring element that is attached to a garment and detachably connected to the controller, the wearable biological signal measuring element comprising:
an electrode configured to come into contact with skin;
an electric line that is connected to the electrode;
an electric connector that is connected to the electric line, penetrates the garment, and is detachably connected to the controller outside the garment; and
a water supply mechanism including a water guiding mechanism including a water guiding portion, the water guiding porting including a first opening outside the garment and a second opening outside the garment, wherein the water guiding portion is configured to be supplied with water from the first opening, and wherein the second opening is substantially in close contact with the electrode.

22. The biological signal measuring device according to claim 21, wherein the wearable biological signal measuring element further comprises a water tank in a middle portion of the water guiding mechanism.

23. The biological signal measuring device according to claim 22, wherein the water guiding mechanism includes an electromagnetic valve between the water tank and the electrode.

24. The biological signal measuring device according to claim 23, wherein a lead-off state is configured to be detected when the electrode is dried, and the electromagnetic valve is configured to be driven to supply water.

25. The biological signal measuring device according to claim 21, wherein a lead-off state is configured to be detected when the electrode is dried.

26. The biological signal measuring device according to claim 21, wherein the electrode is made of a conductive fibrous material.
